(19) United States
(12) Patent Application Publication
(10) Pub. No.: US 2025/0297933 A1
(43) Pub. Date: Sep. 25, 2025

(54) MOLECULAR DETECTION APPARATUS, MOLECULAR DETECTION METHOD, AND MOLECULAR DETECTION SYSTEM

(71) Applicants: KABUSHIKI KAISHA TOSHIBA, Tokyo (JP); TOSHIBA INFRASTRUCTURE SYSTEMS & SOLUTIONS CORPORATION, Kawasaki-shi (JP)

(72) Inventor: Yasushi SHINJO, Kawasaki (JP)

(73) Assignees: KABUSHIKI KAISHA TOSHIBA, Tokyo (JP); TOSHIBA INFRASTRUCTURE SYSTEMS & SOLUTIONS CORPORATION, Kawasaki-shi (JP)

(21) Appl. No.: 19/069,302

(22) Filed: Mar. 4, 2025

(30) Foreign Application Priority Data
Mar. 19, 2024 (JP) 2024-043198

(51) Int. Cl. G01N 5/02

(52) U.S. Cl. CPC G01N 5/02

(57) ABSTRACT

A molecular detection apparatus includes: a detection unit comprising first and second molecular sensors having first and second sensitive films, the first and second molecular sensors being same in detection principle and different in responsiveness; and a processing device to perform calibration using first and second response signals from the first and second molecular sensors. The processing device uses data ΔF_{S} and ΔF_{R} of the first and second response signals which are acquired in a first period when carrier gas not containing the target molecule is supplied to derive a relational expression for approximating ΔF_{S} to a function including ΔF_{R}, approximates ΔF_{S} acquired in a second period when carrier gas containing the target molecule is supplied and the first period according to the relational expression to acquire data ΔF_{SX} of a calibration response signal, and acquire differential data between ΔF_{SX} and ΔF_{S} as data ΔF_{SY} of an apparent response signal.

### CROSS-REFERENCE TO RELATED APPLICATIONS

This application is based upon and claims the benefit of priority from Japanese Patent Applications No. 2024-043198, filed on Mar. 19, 2024; the entire contents of which are incorporated herein by reference.

### FIELD

Embodiments relate to a molecular detection apparatus, a molecular detection method, and a molecular detection system.

### BACKGROUND

A sensing technology using an odor (gas) sensor can digitize smell in the air. This technology is widely used, for example, for odor determination, measurement of volatile organic compounds (VOC) in the atmosphere, performance confirmation of air cleaners, trouble detection of devices, and so on. In recent years, the sensing technology has been increased in interest also in applications for detection of explosives and detection of narcotic drugs and stimulant drugs that have depended on the sense of smell of dogs so far, and for diagnosis of specific diseases based on exhalation, and the like. Therefore, it is desired to enhance the performance of the odor (gas) sensor.

Examples of a conventional gas sensing device include devices such as a flame ionization detector (FID), a photo-ionization detector (PID), and a non-dispersive infra-red (NDIR) gas analyzer. These devices are required to have improved portability (reduction in size and weight), safety, lifetime of a light source, price, and substance recognition, and so on. Specifically, the reduction in size is under development because it is advantageous for incorporation into a processing device and the use at a work site.

A semiconductor gas sensor being a representative of a small-sized sensor can measure a gas concentration by using a change in electrical properties such as electrical resistance that occurs when oxygen adsorbed on metal oxides is consumed by a reducing substance. In recent years, as the metal oxides, many kinds of materials such as tin oxide (SnO₂), zinc oxide (ZnO₂), indium oxide (In₂O₃), tungsten oxide (WO₃), and vanadium oxide (V₂O₃) have been used. Further, it is under development to enhance sensitivity and improve selectivity by introducing an element such as palladium (Pd), platinum (Pt), gold (Au), silver (Ag), or the like into these materials by processing such as doping. However, these efforts have not yet achieved sufficient selectivity or sensitivity.

On the other hand, from a viewpoint of further improvement of sensitivity, selectivity, simplicity, rapidity, reliability, stability, and so on of an odor (gas) sensor, a mass detection-type sensor using a quartz crystal microbalance (QCM), a surface acoustic wave (SAW), a micro cantilever (MCL), or the like, has also been attracting attention in recent years. In the case of the QCM, for example, a sensor has been proposed in which a sensitive film such as an organic polymer adsorbing a target molecule is formed on a surface of the device. When the target molecule is adsorbed on the sensitive film, a mass of the film is increased to change a resonant frequency of a quartz crystal oscillator. The change amount of frequency is in proportion to a mass of an adsorbed analyte molecule, and thus a concentration of the analyte molecule can be measured.

One of the materials of the sensitive film is a metal organic framework (MOF). The MOF is a new porous material that has been studied extensively in recent years. This material is composed of metal ions and an organic ligand connecting the metal ions, and is a structure having a large number of pores with a nanometer size. It is characterized by a large specific surface area of up to 10000 m²/g and a heat resistance exceeding 300° C., and it is expected to be applied to various fields such as gas storage and separation, refining, catalysts, batteries, sensors, and so on.

Conventionally, when introducing the MOF film as a sensitive film for sensor, for example, into a QCM sensor, a drop cast method is used. In the drop cast method, the MOF particles with a submicron order size is synthesized and dispersed in a solvent in advance. However, the particle size of the drop casted MOF film is too large to get the sufficient cohesive force between particles and it leads to problems that the film becomes fragile and peels off and so on. Hence, there is a reported sensor in which a dense thin film with high crystallinity and orientation is formed by using a layer by layer (LBL) method. The LBL is a method in which films are stacked one by one to be grown to get high crystalline monolithic film. However, a MOF type to which this method can be applied is limited. Further, in a case of a high crystalline monolithic film, a path through which the target molecule is adsorbed is only fine pores that exist at the uppermost surface.

Therefore, if the fine pore at the uppermost surface is blocked by a substance such as a water molecule or the like, there is a problem that the sensitivity is lowered extremely.

Hence, there is a proposed method of synthesizing the MOF particles with a nanometer order and forming ink using the synthesized MOF particles to form a uniform and dense sensitive film by a coating method. For example, assuming the QCM sensor, a gas component and odor molecule being detection targets are adsorbed onto the sensitive film, resulting in an increase in mass. In proportion to the increased mass, the resonant frequency of a quartz crystal decreases so that the QCM sensor functions as a gas sensor or an odor sensor.

### BRIEF DESCRIPTION OF THE DRAWINGS

FIG. 1A is a view illustrating an example of a molecular sensor.

FIG. 1B is a view illustrating the example of the molecular sensor.

FIG. 1C is a view illustrating the example of the molecular sensor.

FIG. 2A is a view illustrating another example of the molecular sensor.

FIG. 2B is a view illustrating the other example of the molecular sensor

FIG. 3 is a flowchart illustrating an example of a molecular detection method.

FIG. 4 is a block diagram illustrating an example of a molecular detection apparatus.

FIG. 5 is a block diagram illustrating examples of a detection unit and a processing device included in the molecular detection apparatus.

FIG. 6 is a flowchart illustrating an example of a molecular detection method.

FIG. 7A is a chart supplementing the flowchart illustrating the example of the molecular detection method.

FIG. 7B is a chart supplementing the flowchart illustrating the example of the molecular detection method.

FIG. 7C is a chart supplementing the flowchart illustrating the example of the molecular detection method.

FIG. 7D is a chart supplementing the flowchart illustrating the example of the molecular detection method.

FIG. 8 is a graph illustrating a vibration frequency change amount detected by the molecular detection apparatus.

FIG. 9 is a graph illustrating ΔF_{S} and ΔF_{R}.

FIG. 10 is a graph illustrating an approximate straight line of ΔF_{S}.

FIG. 11 is a graph illustrating ΔF_{SX}.

FIG. 12 is a graph illustrating ΔF_{SY}.

FIG. 13 is a graph illustrating ΔF_{S} and ΔF_{R}.

FIG. 14 is a graph illustrating an approximate straight line of ΔF_{S}.

FIG. 15 is a graph illustrating ΔF_{SX}.

FIG. 16 is a graph illustrating ΔF_{SY}.

FIG. 17 is a graph illustrating ΔF_{S}−ΔF_{R}.

### DETAILED DESCRIPTION

A molecular detection apparatus according to an embodiment is a molecular detection apparatus detectable of a target molecule, including: a detection unit comprising a first molecular sensor having a first sensitive film and a second molecular sensor having a second sensitive film, the first molecular sensor and the second molecular sensor being same as each other in principle of detection of the target molecule and different from each other in responsiveness to the target molecule; and a processing device configured to perform calibration using a first response signal from the first molecular sensor and a second response signal from the second molecular sensor. The processing device is configured to use a data ΔF_{S} of the first response signal and a data ΔF_{R} of the second response signal which are acquired in a first period and derive a relational expression for approximating the ΔF_{S} to a function including the ΔF_{R}, the first period being when a carrier gas not containing the target molecule is supplied to the detection unit, approximate the ΔF_{S} acquired in a second period and the first period according to the relational expression to acquire a data ΔF_{SX} of a calibration response signal, the second period being when the carrier gas containing the target molecule is supplied to the detection unit, and acquire a differential data between the ΔF_{SX} and the ΔF_{S} as a data ΔF_{SY} of an apparent response signal.

Hereinafter, embodiments will be explained with reference to the accompanying drawings. Note that in each of the embodiments, substantially the same constituent parts are denoted by the same reference signs and an explanation thereof will be partly omitted in some cases. The drawings are schematic, and a relation between the thickness and the planar dimension of each part, a thickness ratio between the parts, and so on may differ from actual ones in some cases.

In the following explanation, a “target molecule” refers to a molecule that can be detected by a molecular sensor. The “target molecule” does not necessarily refer to one type of molecule, but may refer to an aggregate of a plurality of types of molecules. The aggregate of the plurality of types of molecules may be, for example, a group of molecules that constitute one type of odor as a whole.

### (Molecular Sensor)

The molecular sensor is applicable to a molecular detection apparatus and a molecular detection system. The molecular sensor according to the embodiment has a sensitive film containing a plurality of MOF particles, and a detection part capable of measuring a change in physical quantity caused due to the adherence of the target molecule to the sensitive film. An average particle size of the MOF particles is preferably 5 nm or more and 100 nm or less from the viewpoint of the film uniformity and film strength, but is not limited to this range. A film thickness of the sensitive film is preferably, but not limited to, 10 nm or more in order to ensure the absolute amount of the MOF and 10 μm or less in order to prevent cracks of the film due to an internal stress or the like.

The sensitive film is analyzed using an analysis method such as energy dispersive X-ray spectroscopy (EDX), by performing a cross-section observation using an optical microscope, a scanning transmission electron microscope (STEM), a transmission electron microscope (TEM), or a scanning electron microscope (SEM). When a metal element is detected by an EDX spectrum, there is a possibility that the MOF contains the metal as its main component, so that it is possible to acquire information regarding a crystal structure based on a higher-resolution high-angle annular dark-field (HAADF)-STEM image or the like, to thereby specify the type of the MOF.

It is preferable that 50% or more of the MOF is zirconium (Zr). This is because a Zr-based one is high both in heat resistance and water resistance. That 50% or more of the MOF is Zr means that the concentration of Zr in the metal element concentration detectable by the EDX is 50% or more.

In particular, it is preferable to use a MOF having a structure (below) in which dicarboxylic acid is coordinated to a hexanuclear Zr₆O₄(OH)₄ cluster. For example, a typical structure is illustrated below.

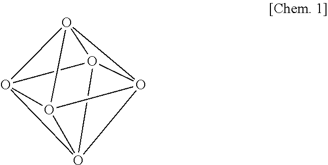

In the above structure, o (white circle) indicates the Zr₆O₄(OH)₄ cluster, and a solid line indicates a dicarboxylic acid ligand.

The MOF having such a structure is at least one of UIO-66, UIO-67, UIO-68, and derivatives thereof. UIO-66, UIO-67, and UIO-68 have structures in which dicarboxylic acid ligands are 1,4-benzenedicarboxylic acid, 4,4′-biphenyldicarboxylic acid, and 4,4″-terphenyldicarboxylic acid, respectively.

The derivatives have new functional groups introduced into benzene rings of the ligands contained in them. Examples of the functional groups include an alkyl group, an amino group, a hydroxy group, an alkoxy group, an amide group, a nitro group, a sulfo group, an aldehyde group, an acyl group, an ester group, a carbonyl group such as a carboxyl group, halogeno groups such as fluorine, chlorine, bromine, iodine, and so on. Examples of the derivatives include a derivative in which the benzene ring of the ligand is substituted with a pyridine ring, an imidazole ring, or a heteroaromatic ring.

Examples of UIO-67 include a material substituted with a polycyclic compound such as 9-fluorenone-2,7-dicarboxylic acid, fluorene-2,7-dicarboxylic acid, or carbazole-2,7-dicarboxylic acid, instead of 4,4′-biphenyldicarboxylic acid.

These MOFs are not only high in heat resistance and high water resistance but also comparatively easy in synthesis and wide in option of the film forming method from fine crystals to thin films, and are easily applicable to the sensitive film. In addition to the above, as MOF containing Zr as its main component, it is possible to use MOF-801, MOF-808, NU-1000, CAU-24, and the like. Further, other than the Zr-based one, it is possible to use MOFs such as MIL-53, MIL-101, MOF-74, and ZIF-8.

The detection part of the molecular sensor may have a measuring mechanism using the QCM, MCL, or SAW.

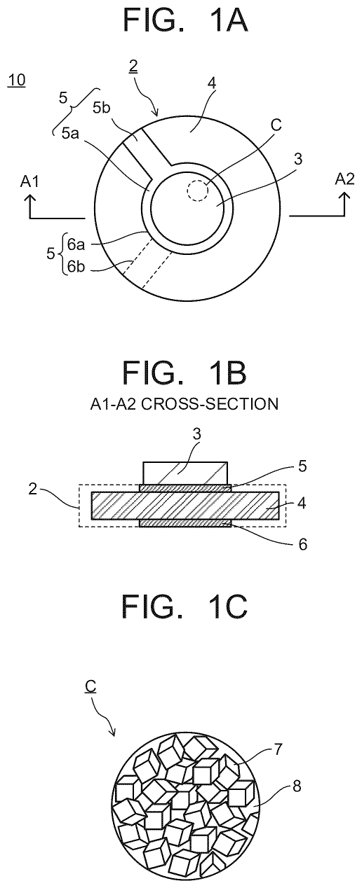

FIG. 1A, FIG. 1B, and FIG. 1C are schematic views illustrating an example of the molecular sensor. FIG. 1A illustrates a plan schematic view of a molecular sensor 10. FIG. 1B illustrates a cross-sectional schematic view taken along a line segment A1-A2 in FIG. 1A. FIG. 1C illustrates an enlarged schematic view of a portion C surrounded by a broken line in FIG. 1A.

The molecular sensor 10 has a measuring mechanism using the QCM as the detection part. The molecular sensor 10 has a QCM detection part 2, and a sensitive film 3 provided on a surface of the QCM detection part 2. The QCM detection part 2 has a disk-shaped base 4, an upper electrode 5, and a lower electrode 6.

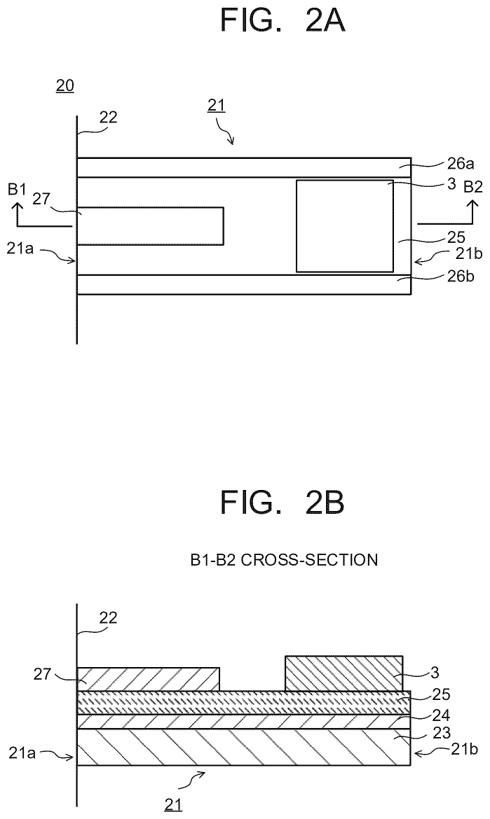

An example of the base 4 includes a quartz crystal substrate. The base 4 is preferably an AT-cut quartz crystal substrate, for example. A planar shape of the base 4 is not limited to the disk shape as illustrated in FIG. 2A, and may be another shape such as a polygon.

The upper electrode 5 is provided on the base 4. As illustrated in FIG. 2A, for example, the upper electrode 5 includes an upper excitation part 5a that is concentric with the base 4 and has a diameter smaller than that of the base 4, and an upper lead-out part 5b that extends from a part of a peripheral edge of the upper excitation part 5a to a peripheral edge of the base 4.

The lower electrode 6 is provided under the base 4. The lower electrode 6 has a lower excitation portion 6a that is concentric with the base 4 and has a diameter smaller than that of the base 4, and a lower lead-out part 6b that extends from a part of a peripheral edge of the lower excitation portion 6a to a peripheral edge of the base 4.

The upper electrode 5 and the lower electrode 6 are, for example, two thin sheet-shaped electrodes that are arranged with the base 4 therebetween. The upper electrode 5 and the lower electrode 6 preferably contain a material such as, for example, platinum (Pt), gold (Au), silver (Ag), copper (Cu), molybdenum (Mo), nickel (Ni), titanium (Ti), tungsten (W), aluminum (Al), indium-tin oxide (ITO), or aluminum-doped zinc oxide (AZO). An example of the upper electrode 5 and the lower electrode 6 includes a stacked film having a Ti layer with a thickness of 10 nm and an Au layer with a thickness of 200 nm provided on the Ti layer. In order to ensure adhesiveness with the sensitive film 3, a Ti layer with a thickness of 10 nm and a silicon oxide (SiO₂) layer with a thickness of 100 nm may be further stacked, as a base layer, on a surface layer of the above Au electrode. The upper electrode 5 and the lower electrode 6 may not have the shapes as illustrated in FIG. 1A, as long as they have shapes capable of exciting the base 4.

The sensitive film 3 has a MOF particle 7. FIG. 1C illustrates a plurality of MOF particles 7. The sensitive film 3 is provided on a surface (upper surface) of the upper excitation part 5a on the opposite side to the base 4, and is provided to cover at least a part of the upper excitation part 5a such that the upper excitation part 5a exists between the sensitive film 3 and the base 4. Not limited to the above, the sensitive film 3 is also provided on the upper side of a surface (lower surface) of the lower excitation portion 6a on the opposite side to the base 4, and is provided to cover at least a part of the lower excitation portion 6a such that the lower excitation portion 6a exists between the sensitive film 3 and the base 4.

For example, when viewed in a plan view, the sensitive film 3 is preferably formed in a disk shape that is concentric with the upper excitation part 5a and has a diameter smaller than that of the upper excitation part 5a. The diameter of the sensitive film 3 is not limited, but is preferably set such that an area of the sensitive film 3 is 20% or more and 90% or less of an area of the base 4, for example.

A dimension of the QCM detection part 2 is not limited in particular, and it may also be similar to that of a general QCM element. For example, a diameter of the base 4 is preferably about 2 mm or more and 10 mm or less.

The molecular sensor 10 may further include an AC power supply that applies a voltage between the upper electrode 5 and the lower electrode 6 via a wire such as a lead wire, and a frequency measuring device that detects a frequency of the base 4. The molecular sensor 10 may also include a temperature adjusting device that heats the sensitive film 3. By heating the sensitive film 3 at a temperature which does not denature the structure of the sensitive film 3, it is possible to remove the adhered target molecule. As a result, a decrease in sensitivity due to the remaining target molecule is prevented, and it becomes possible to perform detection with high sensitivity again.

FIG. 2A and FIG. 2B are schematic views illustrating another example of the molecular sensor. FIG. 2A illustrates a schematic top view of a molecular sensor 20. FIG. 2B illustrates a schematic cross-sectional view taken along B1-B2 of the molecular sensor 20.

The molecular sensor 20 has a measuring mechanism using the MCL as a detection part. The molecular sensor 20 includes an MCL detection part 21 and a sensitive film 3.

The MCL detection part 21 is a long rectangle in a plan view, and has a fixed end 21a fixed to a support 22, and a free end 21b that is not fixed. Specifically, the MCL detection part 21 has a shape of a cantilever. The MCL detection part 21 has a layer structure, and has a substrate 23 provided at the lowermost layer, a lower electrode 24 stacked on the substrate 23, a piezoelectric member 25 stacked on the lower electrode 24, a first upper electrode 26a and a second upper electrode 26b each stacked on the piezoelectric member 25 and extending in a long shape along two long sides, and a detection electrode 27 positioned on the fixed end 21a side between the first upper electrode 26a and the second upper electrode 26b and stacked on the piezoelectric member 25.

The substrate 23 is formed by using a material such as silicon, glass, or resin, for example.

The sensitive film 3 is preferably fixed to a portion close to the free end 21b at the uppermost surface of the MCL detection part 21. The sensitive film 3 can be fixed onto the piezoelectric member 25 between the first upper electrode 26a and the second upper electrode 26b, for example. The MCL detection part 21 may have, between the piezoelectric member 25 and the sensitive film 3, a conductive film such as an Au thin film, an insulating film of SiO₂ or the like, a metal oxide film of aluminum oxide (Al₂O₃), titanium oxide (TiO₂), or the like, or a film of silane coupling agent, a self-assembled monolayer, or the like.

The first upper electrode 26a, the second upper electrode 26b, and the lower electrode 24 are connected to the AC power supply, for example, and can apply the AC voltage to the piezoelectric member 25. The detection electrode 27 can detect a vibration frequency of the piezoelectric member 25.

Each of the first upper electrode 26a, the second upper electrode 26b, the lower electrode 24, and the detection electrode 27 contains a metal material such as, for example, platinum, gold, molybdenum, tungsten, or aluminum. One of the first upper electrode 26a, the second upper electrode 26b, the lower electrode 24, and the detection electrode 27, and another of them may be formed by using mutually different materials.

The piezoelectric member 25 deforms due to the application of voltage, and thus expands and contracts due to the AC voltage to oscillate at a predetermined resonant frequency. The piezoelectric member 25 is formed by using a material such as lead zirconate titanate (PZT), a solid solution of lead zinc niobate-lead titanate (PZN-PT), a solid solution of lead manganate niobate-lead zirconate titanate (PMnN-PZT), aluminum nitride (AlN), zinc oxide (ZnO), potassium sodium niobate (KNN), lithium niobate (LiNbO₃), or the like.

A dimension of the MCL detection part 21 is not limited in particular, and may also be similar to that of a general MCL element. For example, a dimension of the sensitive film 3 when viewed in a plan view can be set to have an area of 20% or more and 90% or less of an area of the MCL 21. The thickness of the sensitive film 3 is preferably, for example, 10 nm or more and 10 μm or less, more preferably 50 nm or more and 5 μm or less.

The molecular sensor 20 can also be used for a detection method similar to that of the molecular sensor 10. When the target molecule adheres to the sensitive film 3 of the molecular sensor 20, an energy loss corresponding to the mass of the target molecule is generated, which changes the resonant frequency of the piezoelectric member 25. This change is measured by the detection electrode 27 to thereby generate an electrical signal. The generated electrical signal is sent to, for example, a later-explained processing device. Consequently, the target molecule can be detected.

The detection part is not limited to the above measuring mechanism, and another measuring mechanism may be used. The detection part may have a mechanism capable of measuring a mass change of the sensitive film 3, for example.

Another example of the detection part may include a measuring mechanism using the SAW. The detection part including the SAW has, for example, two sets of interdigitated electrodes (IDEs) arranged with a desired interval provided therebetween, on a surface of a piezoelectric substrate. The sensitive film 3 can be arranged between the two sets of electrodes on the piezoelectric substrate, for example. When the target molecule adheres to the sensitive film 3, a change occurs in propagation velocity and amplitude of a surface acoustic wave that propagates through the surface of the piezoelectric substrate, and the change is detected by the two electrodes, to thereby generate an electrical signal. The generated electrical signal is sent, for example, to the later-explained processing device. Consequently, the target molecule can be detected.

Another example of the detection part may include a mechanism capable of measuring a change in electrical resistance, impedance, electrical conductivity, or the like of the sensitive film 3, for example. Such a detection part has, for example, a field effect transistor (FET), an IDE-type sensor, or the like. When the detection part has the FET, the sensitive film 3 can function as a channel layer that forms a channel between a source electrode and a drain electrode, for example. When the detection part has the IDE-type sensor, the sensitive film 3 can be provided between or on electrodes of the IDE, for example. For the other explanation of the sensitive film 3, the explanation of the sensitive film 3 in the molecular sensor 10 or the molecular sensor 20 can be cited appropriately.

### (Molecular Detection Method)

Next, a molecule detection method for detecting a target molecule in a sample using a molecular sensor will be explained. Here, the detection can be sensing the type and/or amount of the target molecule, for example.

The sample may be, for example, a solid or liquid capable of generating the target molecule. The solid or liquid sample may generate the target molecule at room temperature and atmospheric pressure. The sample may generate the target molecule when the atmosphere or a carrier gas such as nitrogen or argon is flown or when the sample is heated. Examples of the sample are not limited in particular, and include medicines, foods, drinking water, organisms, fragrances, cargo, luggage, household products, electric appliances, and the like. The sample may be gas. Examples of the gas include atmosphere, exhaled air, exhaust gas, gas fuel, and the like.

The target molecule is, for example, a chemical substance in a gas state. Examples of the target molecule include, but not limited to, VOC, oxygen, hydrogen, carbon dioxide, carbon monoxide, nitrogen, noble gases, hydrogen sulfide, ammonia, nitrogen oxides, acetylene, ethylene, methane, ethane, propane, and the like. The target molecule may be, for example, a chemical substance generated from or contained in narcotics/stimulants, gunpowder, explosives, chemical weapons, fresh food, animals, plants, or the like. The target molecule may be 2-methylisoborneol, geosmin, or the like, which causes a musty odor.

As explained above, the target molecule is not necessarily one type of molecule, and may include a plurality of types of molecules. The plurality of types of molecules may be, for example, a group of molecules that form one odor.

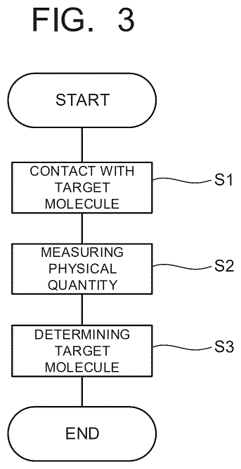

FIG. 3 is a flowchart illustrating an example of the molecular detection method. The example of the molecular detection method has a target molecule contact step S1, a physical quantity measurement step S2, and a target molecule determination step S3 in order. Here, a method for detecting the target molecule using the molecular sensor 10 will be explained as an example.

The target molecule contact step S1 includes bringing the target molecule into contact with the sensitive film 3. At the target molecule contact step S1, first, the molecular sensor 10 is prepared. Next, the target molecule is brought into contact with the sensitive film 3 by performing processing such as placing the molecular sensor 10 in an environment where the same exists, bringing the sample close to the molecular sensor 10, or blowing or sucking air to/from the sample.

By the target molecule contact step S1, the target molecule can adhere to the sensitive film 3. In the case where the target molecule is a molecule likely to adhere to the sensitive film 3, the target molecule can enter from the surface of the sensitive film 3 and penetrate into holes inside the sensitive film 3.

The physical quantity measurement step S2 includes measuring a change in physical quantity of the sensitive film 3 due to the adhesion of the target molecule. The physical quantity can be measured using the QCM detection part 2. The measurement of the physical quantity may be performed by measuring the physical quantity of the sensitive film 3 over time from before the target molecule contact step S1 and measuring the value when the change amount in the measurement value becomes maximum or becomes a constant value (saturation value). The measurement of the physical quantity may be performed by measuring a change rate in the measurement value in a certain period of time. The physical quantity does not need to be measured over time, and only needs to be measured at least at two points in time, that is, before and after the target molecule contact step S1. The physical quantity does not need to be measured before the target molecule contact step S1. In this case, as the measurement value of the physical quantity before the target molecule contact step S1, the measurement value of the physical quantity obtained in the past when the target molecule did not adhere to the sensitive film 3 may be used. As the measurement value of the physical quantity before the target molecule contact step S1, the measurement value of gas not containing a detectable amount of the target molecule, for example, atmosphere may be used.

In the QCM detection part 2, the mass of the sensitive film 3 can be measured by converting the vibration frequency of the base 4 into an electrical signal and detecting it. After the target molecule contact step S1, the vibration frequency of the base 4 changes due to a loss of energy corresponding to the mass of the target molecule adhering to the sensitive film 3, whereby the measurement value of the changed electrical signal can be obtained.

The target molecule determination step S3 includes determining the type or the amount of the target molecule from the result of measurement. At the target molecule determination step S3, the type or the amount of the target molecule can be determined using the result of measurement obtained at the physical quantity measurement step S2. First, the change amount in the measurement value between before and after the target molecule contact step S1 is calculated. For example, the change amount is calculated by subtracting the measurement value before the target molecule contact step S1 from the measurement value after the target molecule contact step S1. The change amount may be used, in the next step, as it is in the change amount in electric signal, or may be used after conversion into the change amount in physical quantity. Next, the type or the amount of the target molecule is determined from the change amount.

For example, by measuring in advance the changes in the physical quantities for a plurality of types of known standard target molecules respectively for a plurality of types of sensitive films 3 before performing the molecular detection method, it is possible to associate the types of detectable target molecules with the types of the sensitive films 3.

Therefore, when the physical quantity in the sample changes using a specific sensitive film 3, comparison with the above association makes it possible to determine which of the standard target molecules detectable by the sensitive film 3 the target molecule contained in the sample is. If the physical quantity does not change in the sample, the detection is repeated again using another sensitive film 3 to find the sensitive film 3 in which the physical quantity changes, whereby the types of target molecules can be narrowed down.

The molecular sensor 10 can also determine the amount and concentration of the target molecule. For example, using the sensitive film 3 associated with a specific standard target molecule, a calibration curve of the change amount of the physical quantity of the sensitive film 3 is created with a plurality of concentrations of the standard target molecule. By comparing the change amount in the sample with this calibration curve, the concentration of the target molecule can be determined.

An example of the molecular detection method may further include a heating removal step of removing the target molecule from sensitive film 3 by heating the sensitive film 3 after the physical quantity measurement step S2. For example, the heating is preferably performed at a temperature of 80° C. or higher and 200° C. or lower for 10 seconds or more and 600 seconds or less. By performing the heating under the conditions, the target molecule that has entered the inside of the sensitive film 3 can be easily released. Since the MOF particles contained in the molecular sensor have excellent heat resistance of, for example, a heat-resistant temperature of 200° C. or higher, almost all the target molecules can be removed without denaturing the structure of the sensitive film 3 even under such heating conditions. As a result, the sensitivity can be prevented from being lowered due to the remaining target molecules, and it becomes possible to bring the molecular sensor 10 into a state of capable of detecting the target molecule with high sensitivity. For this reason, the molecular sensor 10 can be reused by a simple method such as heating and is economical.

### (Molecular Detection Apparatus and Molecular Detection System)

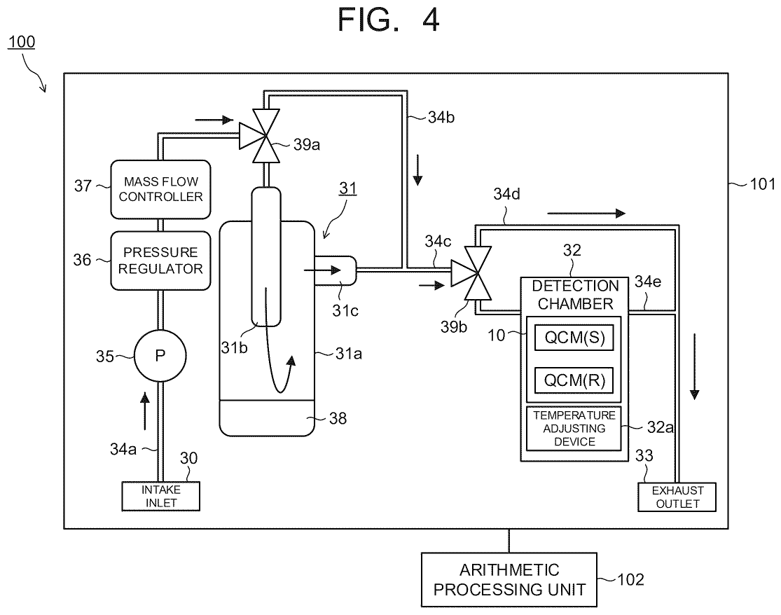

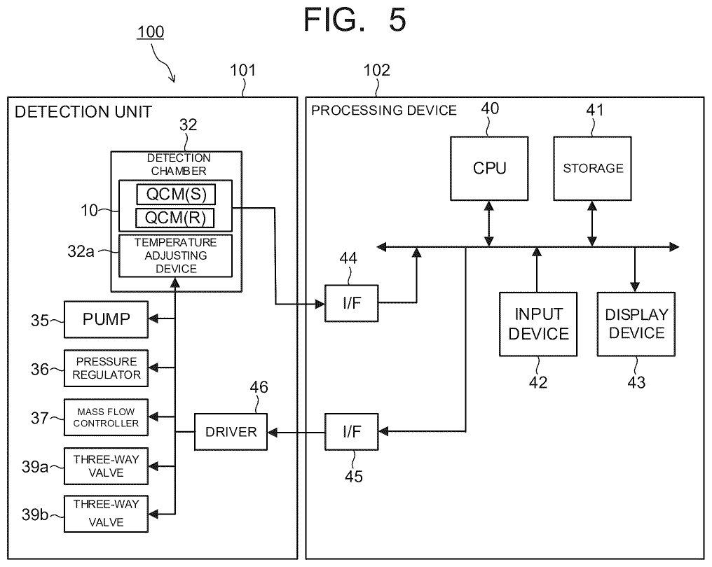

A molecular detection apparatus for detecting the target molecule can be provided using the above molecular sensor. FIG. 4 and FIG. 5 are schematic diagrams illustrating a configuration example of the molecular detection apparatus. FIG. 4 and FIG. 5 illustrate a molecular detection apparatus 100. The molecular detection apparatus 100 has a detection unit 101 and a processing device 102. Note that components of the detection unit 101 and the processing device 102 do not need to be provided in one molecular detection apparatus 100 and may be provided in separate devices to form a molecular detection system.

The detection unit 101 includes an intake inlet 30, a gas generator 31, a detection chamber 32, and an exhaust outlet 33. These elements are connected by, for example, flow paths. The arrows in FIG. 4 represent a direction in which fluid such as gas flows.

The detection unit 101 may have a filter between the intake inlet 30 and a pump 35. The intake inlet 30 and the gas generator 31 are connected by, for example, a flow path 34a. For example, the pump 35, a pressure regulator 36, and a mass flow controller 37 are interposed in the flow path 34a in this order from the intake inlet 30 side toward the gas generator 31.

The processing device 102 may be configured, for example, using hardware that uses a processor and the like. Incidentally, operations may be pre-stored as operating programs in a computer-readable recording medium such as a memory, and the operations may be executed by reading the operating programs stored in the recording medium by the hardware when necessary.

As illustrated in FIG. 5, the processing device 102 includes, for example, a CPU 40, a storage 41, an input device 42, and a display device 43. The processing device 102 may further include an interface (I/F) 44 that outputs a measurement value from the molecular sensor 10 as an electrical signal to the storage 41, and a I/F 45 that outputs a drive signal to the driver 46.

Not limited to the configuration illustrated in FIG. 5, the detection unit 101 may further include the pump 35, the pressure regulator 36, the mass flow controller 37, a three-way valve 39a, a three-way valve 39b, a temperature adjusting device 32a, and a driver 46 that drives a power supply according to a command of the CPU 40.

The gas generator 31 includes, for example, a container 31a for containing a sample 38, a gas introduction pipe 31b for introducing the atmosphere sucked from the intake inlet 30 into the container 31a, a gas lead-out pipe 31c for leading gas out of the container 31a, and a not-illustrated sample feed inlet. The gas introduction pipe 31b is disposed so as to face the sample 38 contained at a bottom of the container 31a, for example. The gas lead-out pipe 31c is connected to the detection chamber 32 by a flow path 34c in which the three-way valve 39b is interposed.

The detection unit 101 has a three-way valve 39a between the mass flow controller 37 and the gas introduction pipe 31b of the gas generator 31. A first valve of the three-way valve 39a is connected to the gas introduction pipe 31b, and a second valve is connected via a flow path 34b to the flow path 34c connecting the gas lead-out pipe 31c and the three-way valve 39b. The detection unit 101 may have a valve for preventing backflow to the gas generator 31, in the middle of the low path 34b and before a connection position between the flow path 34b and the flow path 34c.

A third valve of the three-way valve 39b is connected to one end of the low path 34b. The other end of the flow path 34b is directly connected to the exhaust outlet 33. When performing measurement, the three-way valve 39b is switched to send gas to the detection chamber 32. When not performing measurement, the three-way valve 39b is switched to send gas to a flow path 34d, and thereby can exhaust the gas from the exhaust outlet 33.

The detection chamber 32 houses a sensor unit 1. The sensor unit 1 has a plurality of the molecular sensors 10. The molecular sensor 10 analyzes the target molecule contained in the gas sent from the gas generator 31. The detection chamber 32 may include a temperature adjusting device 32a for heating and cooling the sensitive film 3 of the molecular sensor 10. The detection chamber 32 may further include a power source that supplies a voltage to the detection part of the molecular sensor 10.

The detection chamber 32 is connected to the exhaust outlet 33 by a flow path 34e, so that the analyzed gas is discharged through the flow path 34e.

The detection chamber 32 may be formed, for example, in the shape of a cassette, and installed such that it can be taken into and out of the detection unit 101. In that case, the molecular sensor 10 can be replaced according to the type of the target molecule desired to be detected. The detection chamber 32 may be fixed to the detection unit 101. The molecular sensor 10 disposed in the detection chamber 32 may be any of the above molecular sensors. The molecular sensor may be composed of at least two molecular sensors different in responsiveness to the target molecule, and may be a multi-molecular sensor composed of a plurality of molecular sensors.

The CPU 40 controls the parts of the detection unit 101 and processing device 102 and calculates measurement values according to a program. The storage 41 stores a program to be executed by the CPU 40, information on a measurement value output from the molecular sensor 10, an arithmetic expression and a calibration curve used for calculating the measurement value, and/or a measurement value or a pattern of a standard target molecule. The storage 41 includes a nonvolatile memory such as a flash memory, and a volatile memory such as a RAM.

The input device 42 includes devices such as a keyboard, a mouse, switches, buttons, etc., for inputting various kinds of information to the processing device 102.

The display device 43 includes a device such as a display that displays a calculation result or the like as a chart or text. Instead of separately providing the input device 42 and the display device 43, a touch panel having a display function and an input function may be provided.

The parts in the processing device 102 are connected by, for example, a system bus 47.

The molecular detection apparatus 100 may not include the gas generator 31. In that case, measurement can be performed by bringing the intake inlet 30 close to the sample 38, further blowing and suction, and placing the molecular detection apparatus 100 in a space where the sample 38 exists.

The molecular sensor 10 can be manufactured, for example, as a chip on the order of about 1 mm. The molecular detection apparatus 100 can be manufactured, for example, as a portable device on the order of about several tens of cm.

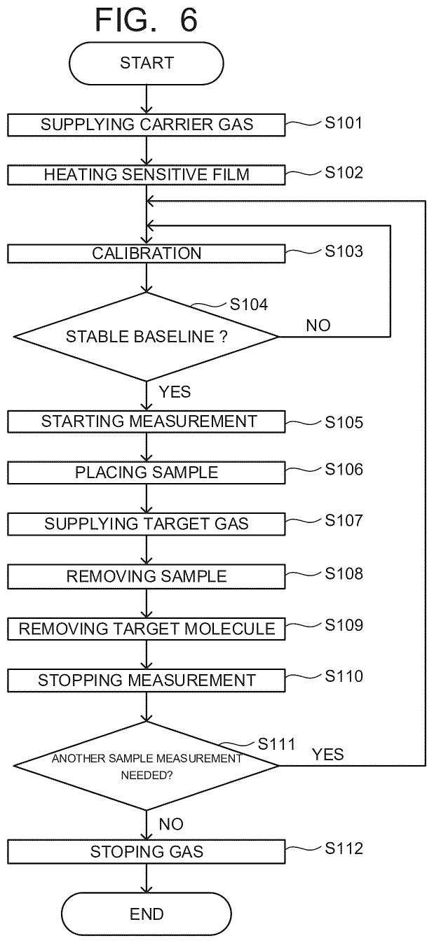

A method of operating the molecular detection apparatus 100 in a molecular detection method example will be explained using FIG. 6. FIG. 6 is a flowchart for explaining an example of the molecular detection method.

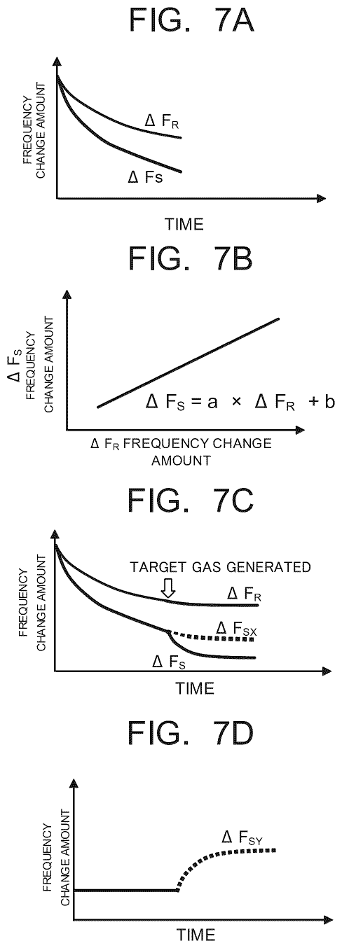

FIG. 7A, FIG. 7B, FIG. 7C, and FIG. 7D are charts for explaining an example of the molecular detection method. The example of the molecular detection method performs carrier gas measurement (FIG. 7A), function fitting (FIG. 7B), target molecule measurement (FIG. 7C), and apparent response signal generation (FIG. 7D) in order. These are performed between the physical quantity measurement step S2 and the target molecule determination step S3.

In the example of the molecular detection method, first, a carrier gas containing no target molecule is supplied to the detection chamber 32 (S101). The carrier gas is, for example, an atmosphere, but may be another gas. As the carrier gas, for example, nitrogen gas, argon gas, or the like connected to, for example, to a container such as a cylinder may be used. When using the cylinder, the cylinder is connected directly to the pressure regulator 36 without using a pump.

For example, according to a command from the CPU 40, the three-way valve 39a is switched to the gas generator 31 side and the three-way valve 39b is switched to the detection chamber 32 side, and the pump 35 is driven through the driver 46. By driving the pump 35, the carrier gas is sent from the intake inlet 30 into the detection chamber 32 through the flow path 34a, the gas generator 31, and the flow path 34c. The mass flow controller 37 controls a flow rate of the carrier gas. By this operation, the gas previously contained in the detection chamber 32 is discharged from the exhaust outlet 33 to the outside.

Next, the sensitive film 3 is heated by driving the temperature adjusting device 32a according to the command of the CPU 40 (S102). Thus, the adsorbed component is removed from the sensitive film 3 which is initialized. Subsequently, the sensitive film 3 is cooled down to the original temperature by the temperature adjusting device 32a. Note that the sensitive film 3 can be initialized only by supplying the carrier gas without performing the heating at Step S102. Then, calibration is performed (S103). If the molecular sensor 10 is a sensor using the QCM, a voltage is applied between the upper electrode 5 and the lower electrode 6, and the frequency measuring device measures the vibration frequency of the base 4. For example, in the case of using two QCMs, they preferably have sensitive films different in property, and the sensitive films are preferably MOFs large in specific surface area and high in adsorption performance.

The plurality of the molecular sensors 10 have, for example, at least a measurement QCM(S) and a reference QCM(R). In the case of the MOF, the measurement QCM(S) and the reference QCM(R) may be different from each other in crystal structure of the MOF particle. The measurement QCM(S) and the reference QCM(R) may be different from each other in at least one of the type of a plurality of metal ions constituting the MOF particle, the type of the organic ligand connecting the plurality of metal ions, and the amount ratio between the plurality of metal ions and the organic ligand. The measurement QCM(S) and the reference QCM(R) may be the same in the type of the plurality of metal ions constituting the MOF particle, the type of the organic ligand connecting the plurality of metal ions, and the amount ratio between the plurality of metal ions and the organic ligand, and different from each other in average particle size of the MOF particle. The measurement QCM(S) and the reference QCM(R) may be the same as each other in the crystal structure of the MOF particle, the type of plurality of metal ions constituting the MOF particle, the type of organic ligand connecting the plurality of metal ions, and the amount ratio between the plurality of metal ions and the organic ligand, and different from each other in average thickness of the sensitive film.

The average thickness of the sensitive film can be measured by using the following method, for example. First, a portion formed with the sensitive film of the molecular sensor is taken out, the sensitive film is checked at a low magnification so as to include the entire sensitive film in a field of view, and the sensitive film is cut in the film thickness direction thereof at a position where specific cracks, defects, projections, foreign matters, and so on do not exist apparently. Next, the sensitive film is subjected to focused ion beam (FIB) machining, and then the cut cross-section is observed. Here, as the FIB apparatus, it is possible to use, for example, SMI3300SE manufactured by Hitachi, Ltd., or Strata 400s manufactured by FEI Company. The observation of the entire film and the observation of the cross-section can be performed by using, for example, the optical microscope, the STEM, the TEM, or the SEM.

After obtaining the cross-section in the film thickness direction of the sensitive film through the above FIB machining, the film thickness of the sensitive film is measured by the above-described STEM or the like. At this time, when selecting an observation portion, the observation of the cross-section is performed at a magnification at which the entire region formed with the sensitive film is included as much as possible in a field of view, and a portion with the largest film thickness is selected. However, a portion where specific cracks, defects, projections, foreign matters, and so on apparently exist when observing the entire film, is not selected. Further, the observation is performed by raising the magnification in a range where the film thickness of the selected portion is included in the field of view.

In the selected respective cross-sections at three locations or more, the film thickness of the sensitive film is measured by the above method. By averaging the film thicknesses of the sensitive film at the respective cross-sections obtained as above, it is possible to calculate the average film thickness of the measured sensitive film 3.

An average particle size of the MOF particle can also be measured by using the image observation of the optical microscope, the STEM, the TEM, or the SEM, in a similar manner to the film thickness measurement of the sensitive film. There are a method of using image analysis software such as ImageJ to extract a contour of the particle, and a method of extracting a contour of the particle based on human judgment while watching an image. Further, by using, in addition to the image observation by the STEM or the like, a mapping image regarding an element (Zr or the like) derived from the component of the MOF at the same cross-section as that captured by the EDX at the time of the film thickness measurement of the sensitive film, it is possible to judge the size and the shape of the particle. Further, in a case of a crystalline substance such as the MOF, it is possible to estimate an average particle size by utilizing a Debye-Scherrer method in which a half value width of a diffraction main peak and a crystal particle size are in a relation of inverse proportion, from a pattern obtained by the X-ray diffraction (XRD).

Concretely, a crystal particle size D (Å) is expressed by D=K/λ(β cos θ). Here, K indicates a constant, and is generally 0.9, although depending on a form factor. β indicates a half value width of diffraction peak (rad), and λ indicates a wavelength of X-ray and is 1.5406 (Å), for example, in a case of CuKa1-ray. θ indicates a Bragg angle (rad).

The conventional molecular sensor using the MOF is affected by a humidity change, a temperature change, an airflow variation, or the like, and therefore may cause a drift phenomenon that the frequency change amount from the start of acquisition of data of the response signal does not become constant but changes with time even in a period when only the carrier gas containing no target molecule is supplied.

Hence, the molecular detection apparatus and the molecular detection system in the embodiments perform calibration using the response signal from the measurement QCM(S) and the response signal from the reference QCM(R). The molecular detection apparatus and the molecular detection system in the embodiments acquire data ΔF_{S} of the response signal from the measurement QCM(S) and data ΔF_{R} of the response signal from the reference QCM(R) for a certain period of time, in a period when only the carrier gas containing no target molecule is supplied, and derive a relational expression established between ΔF_{S} and ΔF_{R} using these pieces of data. For example, by sorting ΔF_{S} and ΔF_{R} acquired in a calibration period in ascending order and approximating ΔF_{S} by linear approximation by least squares method, a linear function of ΔF_{S}≈ΔF_{SX}=a×ΔF_{R}+b (a, b are constants) is obtained. ΔF_{SX} represents data of a calibration response signal of ΔF_{S}. By acquiring ΔF_{SX}, differential data of ΔF_{SX}−ΔF_{S} can be made closer to zero, and a later-explained apparent response signal can be formed. If there is a difference between ΔF_{S} and ΔF_{R} in a period (measurement period) for detecting the target molecule, the differential data ΔF_{SX}−ΔF_{S} is expressed by ΔF_{SX}−ΔF_{S}=f(ΔF_{R})−ΔF_{S}=a×ΔF_{R}+b−ΔF_{S} (a, b are constants), so that the differential data can be acquired as data ΔF_{SY} of the apparent response signal. Note that the calculation processing can be performed, for example, by the CPU 40.

Further, in the calibration period, ΔF_{S} may be approximated by a sum of ΔF_{R} and a time function f(t). For example, when approximating the differential data of ΔF_{S}−ΔF_{R} by linear approximation by least squares method, a linear function of ΔF_{S}−ΔF_{R}≈a×t+b (a, b are constants, t is time) is obtained. In other words, ΔF_{S}≈ΔF_{SX}=a×t+ΔF_{R} (a, b are constants, t is time). Thus, the differential data of ΔF_{SX}−ΔF_{S} can be made closer to zero and the differential data can be acquired as data ΔF_{SY} of the apparent response signal. In this event, if there is a difference between ΔF_{S} and ΔF_{R} in the measurement period, the differential data ΔF_{SX}−ΔF_{S} is expressed by ΔF_{SX}−ΔF_{S}=ΔF_{R}+f(t)−ΔF_{S}=a×t+b+ΔF_{R}−ΔF_{S} (a, b are constants, t is time), so that the differential data can be acquired as ΔF_{SY}. Note that the calculation processing can be performed, for example, by the CPU 40.

As explained above, ΔF_{SX}−ΔF_{S} is acquired as ΔF_{SY}, and the safety of the baseline of the apparent response signal is judged (S104). A method of acquiring ΔF_{SX}−ΔF_{S} as ΔF_{SY} is not particularly limited as long as the safety of the baseline of the apparent response signal can be obtained. Such a baseline correction method effectively functions even for a sensor having a mechanism capable of measuring the change in electrical resistance, impedance, electrical conductivity, or the like other than the above MCL, SAW as well as the QCM. However, a measurement molecular sensor such as the measurement QCM(S) and a reference molecular sensor such as the reference QCM(R) are preferably the same as each other in principle of detection of the target molecule. In the case where the measurement molecular sensor and the reference molecular sensor detect the target molecule by mutually different detection principles, for example, one of the measurement molecular sensor and the reference molecular sensor varies according to the humidity but the other of the measurement molecular sensor and the reference molecular sensor may not vary, and therefore there may be a need to further perform correction for obtaining a correct baseline correction.

If the baseline does not become stable (S104, No), the calibration (S103) is performed again and S103 and S104 are repeated until the baseline becomes stable. The stability of the baseline is judged, for example, by the processing device 102 using a value of the inclination of an approximate straight line of the apparent response signal per unit time. For example, when the absolute value of the inclination of an approximate straight line obtained, for example, by approximating ΔF_{SX}−ΔF_{S} in the calibration period by linear approximation by least squares method is larger than, for example, 1.0×10⁻³ Hz/sec, the baseline is judged to be not stable, whereas when the absolute value is 1.0×10⁻³ Hz/sec or less, the baseline is judged to be stable.

Next, the molecular sensor 10 starts to acquire the differential data ΔF_{SX}−ΔF_{S} acquired at the calibration as the data ΔF_{SY} of the apparent response signal (S105). Next, the sample 38 is contained in the container 31a through the not-illustrated feed inlet of the container 31a of the gas generator 31 (S106). The carrier gas is released from the gas introduction pipe 31b of the gas generator 31 toward the sample 38 in the container 31a, and is discharged from the gas lead-out pipe 31c to the flow path 34c. At that time, the target molecule generated from the sample 38 is also mixed and then discharged together with the carrier gas from the gas lead-out pipe 31c. The carrier gas containing the target molecule, which has flowed into the flow path 34c, is supplied to the detection chamber 32 (S107).

Each of the measurement QCM(S) and the reference QCM(R) is preferably selected so that the absolute value of ΔF_{SX}−ΔF_{S} during the measurement period becomes large as much as possible. Then, the carrier gas containing the target molecule is discharged from the exhaust outlet 33 through the flow path 34e. If the sample 38 cannot be contained in the container 31a, it is only necessary that the three-way valve 39a is switched and set so that the carrier gas flows into the flow path 34b, and the intake inlet 30 is made closer to the sample 38 and sucks the sample 38.

Next, the sample 38 is taken out of the gas generator 31 (S108). As a result, the carrier gas containing no target molecule is supplied to the detection chamber 32. If the target molecule remains in the container 31a, the three-way valve 39a may be switched to the flow path 34b side and thereby can promptly vent the carrier gas to the detection chamber 32.

Next, the target molecule is removed from the sensitive film 3 (S109). The target molecule is likely to be removed by heating the sensitive film 3 by driving the temperature adjusting device 32a, for example, according to the command of the CPU 40. The heated sensitive film 3 is cooled down to the original temperature, for example, by the temperature adjusting device 32a. Note that it is also possible to remove the target molecule from the sensitive film 3 only by supplying the carrier gas without heating. Then, the carrier gas containing the target molecule is discharged from the exhaust outlet 33 through the flow path 34e. Then, the measurement by the molecular sensor is stopped (S110).

After that, when there is another sample desired to be measured (S111, Yes), the process may return to S103 to perform the measurement again. When there is no more sample desired to be measured and the measurement is finished (S111, No), the driving of the pump 35 is stopped, and the supplying the carrier gas is stopped (S112).

On the other hand, after the measurement is finished at S110, the CPU 40 retrieves data on the measurement value and arithmetic expression stored in the storage 41 and calculates a change amount of the measurement value.

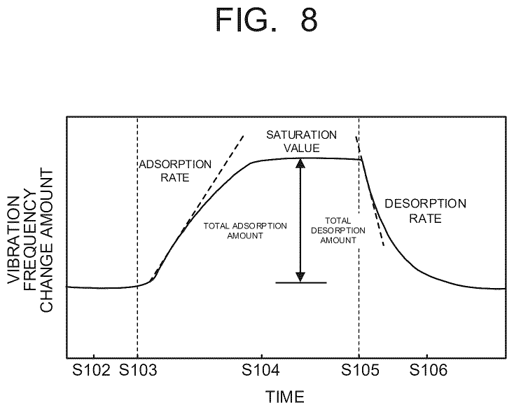

The data to be analyzed may be, as illustrated in FIG. 8, a response amount that can be obtained by subtracting the vibration frequency change amount of the baseline of ΔF_{SX} obtained at S103, S104, S105 from a maximum value or saturation value of the vibration frequency change amount of the apparent response signal obtained at S108. This means a total adsorption amount of the target molecule to the molecular sensor 10.

The data to be analyzed may be an inclination of an approximate straight line at a rising edge of the apparent response signal obtained at S107 or an average inclination of the approximate straight line. This means an adsorption rate of the target molecule to the molecular sensor 10.

The data to be analyzed may be an inclination of an approximate straight line at a falling edge of the apparent response signal obtained at S109 or an average inclination of the approximate straight line. This means a desorption rate of the target molecule from the molecular sensor 10.

The data to be analyzed may be the vibration frequency change amount that can be obtained by subtracting the baseline of the apparent response signal obtained at the end of Step S109 from the maximum value or saturation value of the apparent response signal obtained at S107. This means a total desorption amount of the target molecule from the molecular sensor 10. When the difference from the average value of the differential data between ΔF_{SX} and ΔF_{R} to the maximum value of ΔF_{SX} is defined as a peak height S and a standard deviation σ of the differential data between ΔF_{SX} and ΔF_{R} is defined as a noise width N, an S/N ratio of the apparent response signal is preferably 3 or more, more preferably 5 or more, and furthermore preferably 10 or more.

These pieces of data are output from the CPU 40 to the storage 41 and stored therein. When the detection chamber 32 includes three or more molecular sensors, a pattern of a change amount is further calculated by the CPU 40 for each of the molecular sensors, and output to the storage 41 and stored therein. Subsequently, the CPU 40 retrieves the change amount and the arithmetic expression, if necessary, the calibration curve, the measurement value or the pattern of the standard target molecule, etc., from the storage 41, and determines the type and/or amount of the target molecule. The determination result is output from the CPU 40 to the storage 41 and stored therein. The determination result may be output to the display device 43.

The molecular detection apparatus and the molecular detection system in the embodiments perform calibration using the response signal from the measurement molecular sensor and the response signal from the reference molecular sensor as explained above.

When using the sensitive film large in specific surface area and high in adsorption performance like the MOF film, the sensitive film improves in sensitivity to the target gas or odor but is likely to adsorb also moisture in the atmosphere in many cases, and thus has such a problem that the baseline variation of the response signal obscures a necessary response accompanying a humidity change. The baseline variation may occur due to a temperature change, the influence of airflow to be supplied to the sensor, or the like other than the humidity, so that the measurement with high sensitivity cannot be performed any longer.

In contrast to the above, the molecular detection apparatus and the molecular detection system in the embodiments can correct the baseline of the response signal by performing calibration. Therefore, even the molecular detection apparatus and the molecular detection system using a highly sensitive molecular sensor can decrease the influence of substances other than the target molecule with respect to the response signal, and can realize a molecular detection apparatus, a molecular detection method, and a molecular detection system capable of detecting the target molecule with high sensitivity.

### EXAMPLES

Hereinafter, an example in which the molecular detection apparatus illustrated in FIG. 4 was produced using a QCM molecular sensor formed with the MOF film and the target molecule was detected using the same will be explained.

### Example 1

### [Measurement QCM(S)]

UIO-66 precursor solution was prepared. The UIO-66 precursor solution was prepared by weighting and mixing ZrOCl₂:8H₂O of 18.6 mg, benzene dicarboxylic acid of 9.5 mg, acetic acid of 280 mg, and dimethylformamide of 9.4 g.

A QCM was prepared. The QCM has a resonant frequency of 20 MHz and has, on its one side, a stack composed of a Ti layer with a thickness of 10 nm and a SiO₂ layer with a thickness of 100 nm, as a base layer. The QCM was subjected to ultrasonic cleaning with acetone and pure water, respectively, further dried by a N₂ blower, and then placed at a center of a mini petri dish. An appropriate amount of the prepared precursor solution was dropped to and applied on a surface formed with the SiO₂ layer as an upper surface such that the solution was spread over the whole QCM surface. After that, the QCM was kept standing for 5 hours or more and 6 hours or less. Then, the QCM was taken out and placed on a Teflon (registered trademark) block with a one-side length of 1 cm.

A mixture solution having a volume ratio of acetic acid:dimethylformamide=4:21 was put into a container, and the Teflon block on which the above QCM was mounted was placed on the bottom of the container. The container was put into an oven in a manner to keep the mixture solution in the container out of direct contact with the QCM on the Teflon block, and heated for 3 hours at a temperature of 100° C. Further, the QCM was taken out of the container and heated for 2 hours at a temperature of 150° C. by the hotplate placed in the atmosphere to remove excessive solvent and moisture therefrom. In this manner, the QCM formed with a UIO-66 film was produced to obtain the measurement QCM(S).

### [Reference QCM(R)]

UIO-67 precursor solution was prepared. The precursor solution was prepared by weighting and mixing ZrOCl₂·8H₂O of 18.6 mg, acetic acid of 280 mg, biphenyldicarboxylic acid of 13.9 mg, and dimethylformamide of 9.4 g.

The same method as the production method of the measurement QCM(S) was used other than the use of the thus obtained UIO-67 precursor solution to produce the QCM formed with a UIO-67 film to obtain the reference QCM(R).

### [Measurement]

The produced measurement QCM(S) and reference QCM(R) were installed in the detection chamber 32. Then, the driving of the pump 35 was started, and the pressure regulator 36 and the mass flow controller 37 regulated the flow rate of the carrier gas to 300 ml/min. In this example, the atmosphere introduced from the intake inlet 30 is sent as the carrier gas into the detection chamber 32 through the flow path 34a, the gas generator 31, and the flow path 34c.

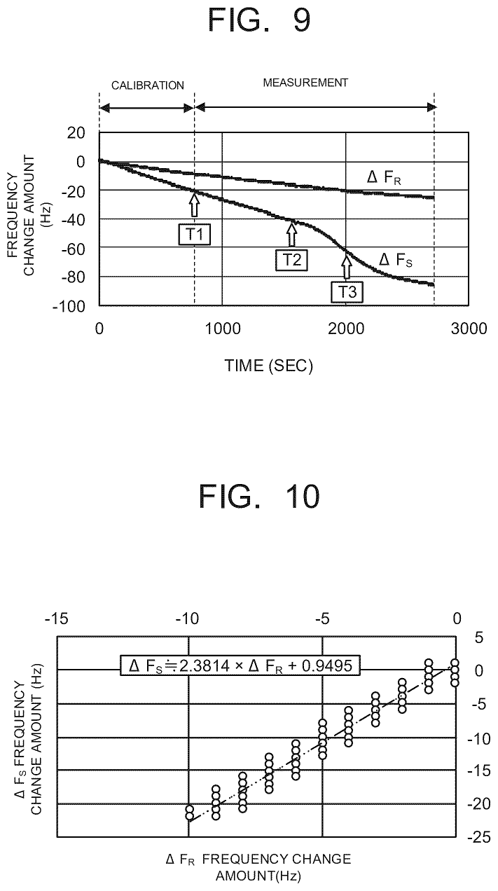

Thereafter, the response signal from the measurement QCM(S) and the response signal from the reference QCM(R) were measured to acquire ΔF_{S} and ΔF_{R} for 800 seconds, calibration was performed using these pieces of data, and then the measurement of ΔF_{S} and ΔF_{R} was continued as they were. The result of this is illustrated in FIG. 9.

At time T1 (800 seconds) illustrated in FIG. 9, a polyethylene container containing 2-MIB powder with a lid closed was placed as the container 31a, and slight odor penetrating through the container was measured by measuring ΔF_{S} and ΔF_{R} by the measurement QCM(S) and the reference QCM(R). In this event, the measurement value of a PID concentration meter connected to the exhaust outlet 33 illustrated in FIG. 4 remained at 0 ppbv. Further, by another experiment, the gas generated under the same conditions was sampled, and it was found as a result of a quantitative analysis that gas of about 0.2 ppbv was generated.

Then, at time T2 (1600 seconds) illustrated in FIG. 9, the polyethylene container was placed with the lid opened. In this event, the measurement value of the PID concentration meter was 10 ppbv or more and 20 ppbv or less. Further, by another experiment, the gas generated under the same conditions was sampled, and it was found as a result of a quantitative analysis that gas of about 18 ppbv was generated.

Further, at time T3 (2000 seconds), the polyethylene container was taken out, and the generation of odor was finished. In this event, the measurement value of the PID concentration meter decreased down to 0 ppbv or more and 5 ppbv or less.

In a period from 0 seconds to 800 seconds (calibration period), the frequency change amount of the response signal from each of the measurement QCM(S) and the reference QCM(R) tended to decrease gradually. This is considered because the mass increased due to gradual adsorption of the moisture in the air to the sensitive films of the measurement QCM(S) and the reference QCM(R). Further, the rate of decrease in frequency change amount tended to be always larger in the measurement QCM(S) than in the reference QCM(R).

At time T1, there was no visually recognizable change in the frequency change amount both in ΔF_{R} and ΔF_{S}. At time T2, there was no visually recognizable change in the frequency change amount in ΔF_{R}, but the frequency change amount in ΔF_{S} became clearly large after time T2. Thereafter, at time T3, there was still no noticeable change in the frequency change amount in ΔF_{R}, but the frequency change amount in ΔF_{S} tended to gradually decrease after time T3.

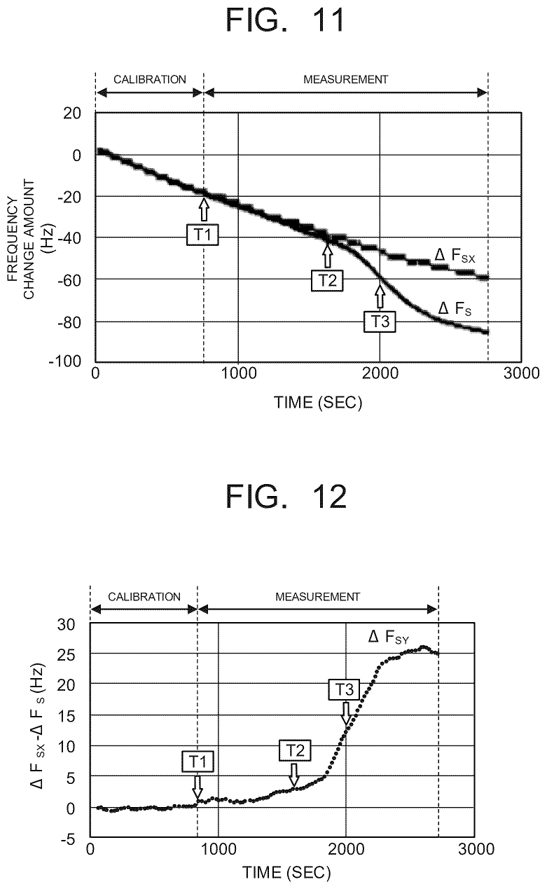

As a result of sorting the acquired ΔF_{S} and ΔF_{R} in ascending order and approximating them by linear approximation by least squares method in the period from 0 seconds to 800 seconds, a relational expression expressed by ΔF_{S}≈ΔF_{SX}=2.3814×ΔF_{R}+0.9495 was derived as illustrated in FIG. 10. In this event, an R-squared value was 0.9715. ΔF_{S} and ΔF_{R} in the period from 0 seconds to 800 seconds and ΔF_{S} in a period (measurement period) after 800 seconds to the end of the measurement were approximated according to the derived relational expression to acquire approximate data (ΔF_{SX}) of ΔF_{S}, and the acquired ΔF_{SX} was arranged to generate a calibration response signal, which is illustrated in FIG. 11. Further, ΔF_{SX}−ΔF_{S} was obtained as ΔF_{SY} and arranged to generate an apparent response signal, which is illustrated in FIG. 12. Note that a moving average deviation for 60 seconds is plotted in FIG. 12.

When the inclination of the approximate straight line obtained by approximating the apparent response signal in the period from 0 seconds to 800 seconds by linear approximation by least squares method was calculated, the inclination was about 6.98×10⁻⁴ Hz/sec, and stabilized near about zero.

It is considered that at time T1 (800 seconds), 2-MIB gas of about 0.2 ppbv was considered to have been generated, but when the inclination of the approximate straight line obtained by approximating the apparent response signal in the period from 800 seconds to 1600 seconds by linear approximation by least squares method was calculated, the inclination (frequency change rate) was about 2.5×10⁻³ Hz/sec, and tended to gradually increase. The reason why the gradual inclination can be recognized is that the inclination of the approximate straight line of the apparent response signal in the period from 0 seconds to 800 seconds is sufficiently small such as 6.98×10⁻⁴ Hz/sec. The inclination of the approximate straight line of the apparent response signal in the calibration period is preferably ⅓ or less of the inclination of the approximate straight line of the apparent response signal in the measurement period, and its absolute value is preferably 1.0×10⁻³ Hz/sec or less. As illustrated in FIG. 12, near time (1600 seconds) when the gas of about 18 ppbv was considered to be generated, there was less change in the apparent response signal at the beginning but a clear rise was confirmed in the apparent response signal after several hundreds of seconds. The average frequency change rate of the apparent response signal in a period from 1800 seconds (time T2) at the start of rising to 2000 seconds (time T3) was 4.5×10⁻² Hz/sec. Thereafter, at the time point of 2000 seconds (time T3) after the finish of the generation of odor, the apparent response signal continuously changed due to the influence of the odor remaining in the piping or the like, but the change in the apparent response signal tended to gradually decrease after several hundreds of seconds and saturate.

### Example 2

As in Example 1, UIO-66 was employed for the measurement QCM(S) and UIO-67 was employed for the reference QCM(R), and their film thicknesses were appropriately adjusted to form films.

The produced measurement QCM(S) and reference QCM(R) were installed in the detection chamber 32. Then, the driving of the pump 35 was started, and the pressure regulator 36 and the mass flow controller 37 regulated the flow rate of the carrier gas to 50 ml/min. In this example, the atmosphere introduced from the intake inlet 30 is sent into the detection chamber 32 through the flow path 34a, the gas generator 31, and the flow path 34c.

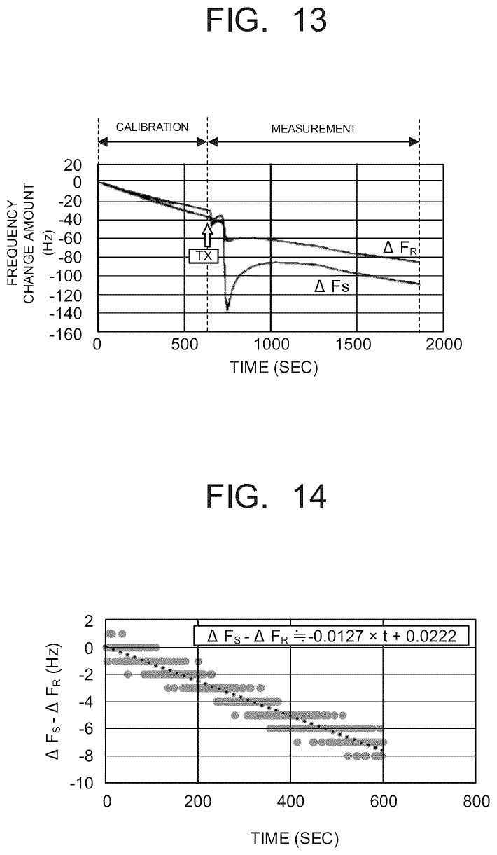

Thereafter, ΔF_{S} and ΔF_{R} were acquired for 600 seconds, calibration was performed using these pieces of data, and then the measurement of ΔF_{S} and ΔF_{R} was continued as they were. The result of this is illustrated in FIG. 13.

At time TX (600 seconds) illustrated in FIG. 13, a cap of a permanent marker was placed in the container 31a. In FIG. 13, in a period from 0 seconds to 600 seconds (calibration period), the frequency change amount of each of ΔF_{R} and ΔF_{S} tended to decrease gradually. This is considered because the mass increased due to gradual adsorption of the moisture in the air to the sensitive films of the measurement QCM(S) and the reference QCM(R). Further, the rate of decrease in frequency change amount was not largely different between ΔF_{R} and ΔF_{S}. At time TX, both of the frequency change amount of ΔF_{R} and the frequency change amount of ΔF_{S} once slightly changed in a negative direction immediately after odor was generated. Thereafter, both of the reference QCM(R) and the measurement QCM(S) responded, and the responsiveness (frequency change amount) of the measurement QCM(S) was clearly larger.

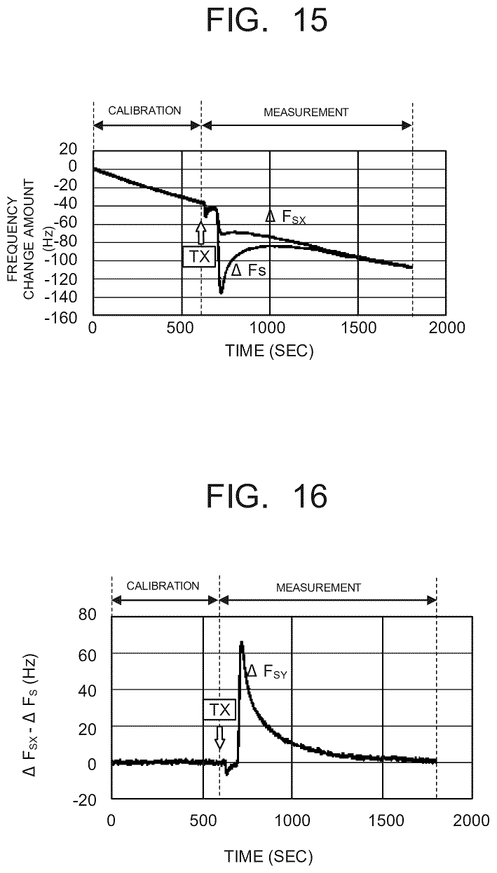

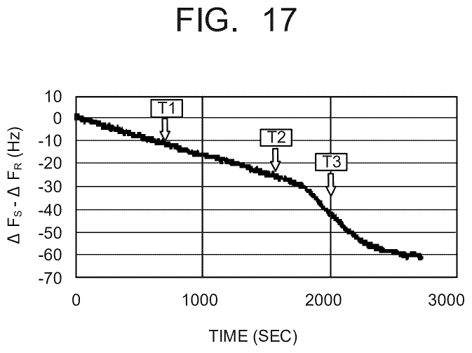

As a result of plotting temporal changes of ΔF_{S}−ΔF_{R} in the calibration period, illustrating them in FIG. 14, and approximating them by linear approximation by least squares method, a relational expression expressed by ΔF_{S}−ΔF_{R}≈−0.0127×t+0.0222 (t is time) was derived. This can be converted into ΔF_{S}≈ΔF_{SX}=−0.0127×t+0.0222+ΔF_{R}. In this event, an R-squared value was 0.931. ΔF_{S} in the calibration period and ΔF_{S} in a period (measurement period) from 500 seconds to the end of the measurement were approximated according to the relational expression to acquire ΔF_{SX}, and the acquired ΔF_{SX} was arranged to generate a calibration response signal, which is illustrated in FIG. 15. ΔF_{SX}−ΔF_{S} was acquired as ΔF_{SY} and arranged to generate an apparent response signal, which is illustrated in FIG. 16.

When the inclination of an approximated curve obtained by approximating the apparent response signal in the period from 0 seconds to 600 seconds by linear approximation by least squares method was calculated, the inclination was about 6.01×10⁻¹⁸ Hz/sec, and stabilized near about zero.

In ΔF_{S} and ΔF_{R}, immediately after time TX, there was a slight response that the frequency change amount decreased once in a negative direction as illustrated in FIG. 15, but there was a response that the frequency change amount rapidly increased thereafter and immediately attenuated after reaching peaks. In Example 2, a small amount of solvent (type unknown) released from the cap of the permanent marker was measured and its volatility was high, and therefore it is considered that the concentration immediately decreased. ΔF_{S} and ΔF_{R} attenuated in response while finally gradually approaching near zero of the baseline, and therefore it was found that the apparent response signal illustrated in FIG. 16 was correctly acquired.

### Comparative Example 1

ΔF_{S} and ΔF_{R} were measured under the same conditions as those of Example 1, ΔF_{S} and ΔF_{R} were used as they were without generating ΔF_{SX} by calibration, and a response signal was generated from differential data ΔF_{S}−ΔF_{R} between ΔF_{S} and ΔF_{R}, which is illustrated in FIG. 17. When the inclination of the approximate straight line obtained by approximating the apparent response signal in the period from 0 seconds to 800 seconds by linear approximation by least squares method was calculated, the inclination was-0.0168 Hz/sec, and the absolute value was 0.0168 Hz/sec=1.68×10⁻². It is also found that the response signal illustrated in FIG. 17 becomes entirely larger in inclination of an approximated curve than the apparent response signals illustrated in FIG. 12 and FIG. 16. It is also found that the apparent response signal has inflection points at time T2 (1600 seconds) when gas of about 18 ppbv was considered to be generated and at time T3 (2000 seconds) when the generation of the gas was considered to end similarly to the apparent response signal illustrated in FIG. 12, but the frequency change amount of the response signal was small before and after time T1 (800 seconds) when gas of about 0.2 ppbv was considered to be generated, and it was difficult to confirm any response.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

The above-described embodiments can be summarized into the following clauses.

### (Clause 1)

A molecular detection apparatus for detecting a target molecule, including:
a detection unit comprising a first molecular sensor having a first sensitive film and a second molecular sensor having a second sensitive film, the first molecular sensor and the second molecular sensor being same as each other in principle of detection of the target molecule and different from each other in responsiveness to the target molecule; and
a processing device configured to perform calibration using a first response signal from the first molecular sensor and a second response signal from the second molecular sensor, wherein
the processing device is configured to
   use a data ΔF_{S} of the first response signal and a data ΔF_{R} of the second response signal which are acquired in a first period and derive a relational expression for approximating the ΔF_{S} to a function including the ΔF_{R}, the first period being when a carrier gas not containing the target molecule is supplied to the detection unit,
   approximate the ΔF_{S} acquired in a second period and the first period according to the relational expression to acquire a data ΔF_{SX} of a calibration response signal, the second period being when the carrier gas containing the target molecule is supplied to the detection unit, and
   acquire a differential data between the ΔF_{SX} and the ΔF_{S} as a data ΔF_{SY} of an apparent response signal.

### (Clause 2)

The molecular detection apparatus according to Clause 1, wherein
the processing device is configured to
derive a first relational expression for approximating the ΔF_{S} to a linear function f(ΔF_{R}) of the ΔF_{R}, and
approximate the ΔF_{S} according to the first relational expression to acquire the ΔF_{SX}.

### (Clause 3)

The molecular detection apparatus according to Clause 1, wherein
the processing device is configured to
derive a second relational expression for approximating the ΔF_{S} to a sum ΔF_{R}+f(t) (t is time) of the ΔF_{R} and a time function, and
approximate the ΔF_{S} according to the second relational expression to acquire the ΔF_{SX}.

### (Clause 4)

The molecular detection apparatus according to any one of Clause 1 to Clause 3, wherein
when a difference from an average value of the differential data between the ΔF_{SX} and the ΔF_{R} to a maximum value of the ΔF_{SX} is defined as a peak height S and a standard deviation σ of the differential data between the ΔF_{SX} and the ΔF_{R} is defined as a noise width N, an S/N ratio of the apparent response signal is 3 or more.

### (Clause 5)

The molecular detection apparatus according to any one of Clause 1 to Clause 4, wherein
an inclination of a first approximate straight line of the apparent response signal in the first period is ⅓ or less of an inclination of a second approximate straight line of the apparent response signal in the second period.

### (Clause 6)

The molecular detection apparatus according to any one of Clause 1 to Clause 5, wherein:
the first sensitive film has a first metal organic framework particle;
the second sensitive film has a second metal organic framework particle; and
an average particle size of each of the first metal organic framework particle and the second metal organic framework particle is 5 nm or more and 100 nm or less.

### (Clause 7)

The molecular detection apparatus according to any one of Clause 1 to Clause 6, wherein:
an average thickness of the first sensitive film is 10 nm or more and 10 μm or less; and
an average thickness of the second sensitive film is 10 nm or more and 10 μm or less.

### (Clause 8)

The molecular detection apparatus according to any one of Clause 1 to Clause 7, wherein:
the first sensitive film has a first metal organic framework;
the second sensitive film has a second metal organic framework; and
50% or more of each of the first metal organic framework and the second metal organic framework is zirconium.

### (Clause 9)

The molecular detection apparatus according to any one of Clause 1 to Clause 7, wherein:
the first sensitive film has a first metal organic framework;
the second sensitive film has a second metal organic framework; and
each of the first metal organic framework and the second metal organic framework has a structure in which dicarboxylic acid is coordinated to a hexanuclear Zr₆O₄(OH)₄ cluster.

### (Clause 10)

The molecular detection apparatus according to any one of Clause 1 to Clause 7, wherein:
the first sensitive film has a first metal organic framework;
the second sensitive film has a second metal organic framework; and
each of the first metal organic framework and the second metal organic framework contains at least one selected from the group consisting of UIO-66, UIO-67, UIO-68, and derivatives thereof.

### (Clause 11)

The molecular detection apparatus according to any one of Clause 1 to Clause 10, further including
a temperature adjusting device configured to heat the first sensitive film and the second sensitive film.

### (Clause 12)

The molecular detection apparatus according to any one of Clause 1 to Clause 11, wherein
each of the first molecular sensor and the second molecular sensor includes a measuring mechanism using a quartz crystal microbalance, a measuring mechanism using a micro cantilever, or a measuring mechanism using a surface acoustic wave.

### (Clause 13)

The molecular detection apparatus according to any one of Clause 1 to Clause 12, wherein:
the first sensitive film has a first metal organic framework particle;
the second sensitive film has a second metal organic framework particle; and
the first metal organic framework particle and the second metal organic framework particle are different from each other in crystal structure.

### (Clause 14)

The molecular detection apparatus according to any one of Clause 1 to Clause 12, wherein:
the first sensitive film has a first metal organic framework particle;
the second sensitive film has a second metal organic framework particle;
each of the first metal organic framework particle and the second metal organic framework particle contains a plurality of metal ions and an organic ligand connecting the plurality of metal ions;
the first metal organic framework particle and the second metal organic framework particle are same as each other in crystal structure; and
the first metal organic framework particle and the second metal organic framework particle are different from each other in at least one selected from the group consisting of type of the metal ion, type of the organic ligand, and amount ratio between the metal ion and the organic ligand.

### (Clause 15)

The molecular detection apparatus according to any one of Clause 1 to Clause 12, wherein:
the first sensitive film has a first metal organic framework particle;
the second sensitive film has a second metal organic framework particle;
each of the first metal organic framework particle and the second metal organic framework particle contains a plurality of metal ions and an organic ligand connecting the plurality of metal ions;
the first metal organic framework particle and the second metal organic framework particle are same as each other in crystal structure;
the first metal organic framework particle and the second metal organic framework particle are same as each other in type of the metal ion, type of the organic ligand, and amount ratio between the metal ion and the organic ligand; and
the first metal organic framework particle and the second metal organic framework particle are different from each other in average particle size.

### (Clause 16)

The molecular detection apparatus according to any one of Clause 1 to Clause 12, wherein:
the first sensitive film has a first metal organic framework particle;
the second sensitive film has a second metal organic framework particle;
each of the first metal organic framework particle and the second metal organic framework particle contains a plurality of metal ions and an organic ligand connecting the plurality of metal ions;
the first metal organic framework particle and the second metal organic framework particle are same as each other in type of the metal ion, type of the organic ligand, and amount ratio between the metal ion and the organic ligand; and
the first sensitive film and the second sensitive film are different from each other in average thickness.

### (Clause 17)

A molecular detection method using a molecular detection apparatus for detecting a target molecule,
the molecular detection apparatus comprising:
   a detection unit comprising a first molecular sensor having a first sensitive film and a second molecular sensor having a second sensitive film, the first molecular sensor and the second molecular sensor being same as each other in principle of detection of the target molecule and different from each other in responsiveness to the target molecule; and
   a processing device configured to perform calibration using a first response signal from the first molecular sensor and a second response signal from the second molecular sensor,
the molecular detection method comprising:
using a data ΔF_{S} of the first response signal and a data ΔF_{R} of the second response signal which are acquired in a first period when carrier gas not containing the target molecule is supplied to the detection unit to derive a relational expression for approximating the ΔF_{S} to a function including the ΔF_{R};
approximating the ΔF_{S} acquired in a second period when carrier gas containing the target molecule is supplied to the detection unit and the first period according to the relational expression to acquire data ΔF_{SX} of a calibration response signal; and
acquiring differential data between the ΔF_{SX} and the ΔF_{S} as data ΔF_{SY} of an apparent response signal.

### (Clause 18)

The molecular detection method according to Clause 17, further including
after acquiring the ΔF_{S} in the second period, heating the first sensitive film to remove the target molecule from the first sensitive film.

### (Clause 19)

A molecular detection system for detecting a target molecule, including:
a plurality of molecular sensors including a first molecular sensor having a first sensitive film and a second molecular sensor having a second sensitive film, the first molecular sensor and the second molecular sensor being same as each other in principle of detection of the target molecule and different from each other in responsiveness to the target molecule; and
a processing device configured to perform calibration using a first response signal from the first molecular sensor and a second response signal from the second molecular sensor, wherein
the processing device is configured to
   use a data ΔF_{S} of the first response signal and a data ΔF_{R} of the second response signal which are acquired in a first period and derive a relational expression for approximating the ΔF_{S} to a function including the ΔF_{R}, the first period being when carrier gas not containing the target molecule is supplied to the plurality of the molecular sensors;
   approximate the ΔF_{S} acquired in a second period and the first period according to the relational expression to acquire a data ΔF_{SX} of a calibration response signal, the second period being when carrier gas containing the target molecule is supplied to the plurality of the molecular sensors; and
   acquire a differential data between the ΔF_{SX} and the ΔF_{S} as data ΔF_{SY} of an apparent response signal.

What is claimed is:

1. A molecular detection apparatus detectable of a target molecule, comprising:
a detection unit comprising a first molecular sensor having a first sensitive film and a second molecular sensor having a second sensitive film, the first molecular sensor and the second molecular sensor being same as each other in principle of detection of the target molecule and different from each other in responsiveness to the target molecule; and
a processing device configured to perform calibration using a first response signal from the first molecular sensor and a second response signal from the second molecular sensor, wherein
the processing device is configured to
use a data ΔF_{S} of the first response signal and a data ΔF_{R} of the second response signal which are acquired in a first period and derive a relational expression for approximating the ΔF_{S} to a function including the ΔF_{R}, the first period being when a carrier gas not containing the target molecule is supplied to the detection unit,
approximate the ΔF_{S} acquired in a second period and the first period according to the relational expression to acquire a data ΔF_{SX} of a calibration response signal, the second period being when the carrier gas containing the target molecule is supplied to the detection unit, and
acquire a differential data between the ΔF_{SX} and the ΔF_{S} as a data ΔF_{SY} of an apparent response signal.

2. The molecular detection apparatus according to claim 1, wherein
the processing device is configured to
derive a first relational expression for approximating the ΔF_{S} to a linear function f(ΔF_{R}) of the ΔF_{R}, and
approximate the ΔF_{S} according to the first relational expression to acquire the ΔF_{SX}.

3. The molecular detection apparatus according to claim 1, wherein
the processing device is configured to
derive a second relational expression for approximating the ΔF_{S} to a sum ΔF_{R}+f(t) (t is time) of the ΔF_{R} and a time function, and
approximate the ΔF_{S} according to the second relational expression to acquire the ΔF_{SX}.

4. The molecular detection apparatus according to claim 1, wherein
when a difference from an average value of the differential data between the ΔF_{SX} and the ΔF_{R} to a maximum value of the ΔF_{SX} is defined as a peak height S and a standard deviation σ of the differential data between the ΔF_{SX} and the ΔF_{R} is defined as a noise width N, an S/N ratio of the apparent response signal is 3 or more.

5. The molecular detection apparatus according to claim 1, wherein
an inclination of a first approximate straight line of the apparent response signal in the first period is ⅓ or less of an inclination of a second approximate straight line of the apparent response signal in the second period.

6. The molecular detection apparatus according to claim 1, wherein:
the first sensitive film has a first metal organic framework particle;
the second sensitive film has a second metal organic framework particle; and
an average particle size of each of the first metal organic framework particle and the second metal organic framework particle is 5 nm or more and 100 nm or less.

7. The molecular detection apparatus according to claim 1, wherein:
an average thickness of the first sensitive film is 10 nm or more and 10 μm or less; and
an average thickness of the second sensitive film is 10 nm or more and 10 μm or less.

8. The molecular detection apparatus according to claim 1, wherein:
the first sensitive film has a first metal organic framework;
the second sensitive film has a second metal organic framework; and
50% or more of each of the first metal organic framework and the second metal organic framework is zirconium.

9. The molecular detection apparatus according to claim 1, wherein:
the first sensitive film has a first metal organic framework;
the second sensitive film has a second metal organic framework; and
each of the first metal organic framework and the second metal organic framework has a structure in which dicarboxylic acid is coordinated to a hexanuclear Zr₆O₄(OH)₄ cluster.

10. The molecular detection apparatus according to claim 1, wherein:
the first sensitive film has a first metal organic framework;
the second sensitive film has a second metal organic framework; and
each of the first metal organic framework and the second metal organic framework contains at least one selected from the group consisting of UIO-66, UIO-67, UIO-68, and derivatives thereof.

11. The molecular detection apparatus according to claim 1, further comprising
a temperature adjusting device configured to heat the first sensitive film and the second sensitive film.

12. The molecular detection apparatus according to claim 1, wherein
each of the first molecular sensor and the second molecular sensor includes a measuring mechanism using a quartz crystal microbalance, a measuring mechanism using a micro cantilever, or a measuring mechanism using a surface acoustic wave.

13. The molecular detection apparatus according to claim 1, wherein:
the first sensitive film has a first metal organic framework particle;
the second sensitive film has a second metal organic framework particle; and
the first metal organic framework particle and the second metal organic framework particle are different from each other in crystal structure.

14. The molecular detection apparatus according to claim 1, wherein:
the first sensitive film has a first metal organic framework particle;
the second sensitive film has a second metal organic framework particle;
each of the first metal organic framework particle and the second metal organic framework particle contains a plurality of metal ions and an organic ligand connecting the plurality of metal ions;
the first metal organic framework particle and the second metal organic framework particle are same as each other in crystal structure; and
the first metal organic framework particle and the second metal organic framework particle are different from each other in at least one selected from the group consisting of type of the metal ion, type of the organic ligand, and amount ratio between the metal ion and the organic ligand.

15. The molecular detection apparatus according to claim 1, wherein:
the first sensitive film has a first metal organic framework particle;
the second sensitive film has a second metal organic framework particle;
each of the first metal organic framework particle and the second metal organic framework particle contains a plurality of metal ions and an organic ligand connecting the plurality of metal ions;
the first metal organic framework particle and the second metal organic framework particle are same as each other in crystal structure;
the first metal organic framework particle and the second metal organic framework particle are same as each other in type of the metal ion, type of the organic ligand, and amount ratio between the metal ion and the organic ligand; and
the first metal organic framework particle and the second metal organic framework particle are different from each other in average particle size.

16. The molecular detection apparatus according to claim 1, wherein:
the first sensitive film has a first metal organic framework particle;
the second sensitive film has a second metal organic framework particle;
each of the first metal organic framework particle and the second metal organic framework particle contains a plurality of metal ions and an organic ligand connecting the plurality of metal ions;
the first metal organic framework particle and the second metal organic framework particle are same as each other in type of the metal ion, type of the organic ligand, and amount ratio between the metal ion and the organic ligand; and
the first sensitive film and the second sensitive film are different from each other in average thickness.

17. A molecular detection method of detecting a target molecule using a molecular detection apparatus,
the molecular detection apparatus comprising:
a detection unit comprising a first molecular sensor having a first sensitive film and a second molecular sensor having a second sensitive film, the first molecular sensor and the second molecular sensor being same as each other in principle of detection of the target molecule and different from each other in responsiveness to the target molecule; and
a processing device configured to perform calibration using a first response signal from the first molecular sensor and a second response signal from the second molecular sensor,
the molecular detection method comprising:
using a data ΔF_{S} of the first response signal and a data ΔF_{R} of the second response signal which are acquired in a first period when carrier gas not containing the target molecule is supplied to the detection unit to derive a relational expression for approximating the ΔF_{S} to a function including the ΔF_{R};
approximating the ΔF_{S} acquired in a second period when carrier gas containing the target molecule is supplied to the detection unit and the first period according to the relational expression to acquire data ΔF_{SX} of a calibration response signal; and
acquiring differential data between the ΔF_{SX} and the ΔF_{S} as data ΔF_{SY} of an apparent response signal.

18. The molecular detection method according to claim 17, further comprising
after acquiring the ΔF_{S} in the second period, heating the first sensitive film to remove the target molecule from the first sensitive film.

19. A molecular detection system detectable of a target molecule, comprising:
a plurality of molecular sensors including a first molecular sensor having a first sensitive film and a second molecular sensor having a second sensitive film, the first molecular sensor and the second molecular sensor being same as each other in principle of detection of the target molecule and different from each other in responsiveness to the target molecule; and
a processing device configured to perform calibration using a first response signal from the first molecular sensor and a second response signal from the second molecular sensor, wherein
the processing device is configured to
use a data ΔF_{S} of the first response signal and a data ΔF_{R} of the second response signal which are acquired in a first period and derive a relational expression for approximating the ΔF_{S} to a function including the ΔF_{R}, the first period being when carrier gas not containing the target molecule is supplied to the plurality of the molecular sensors;
approximate the ΔF_{S} acquired in a second period and the first period according to the relational expression to acquire a data ΔF_{SX} of a calibration response signal, the second period being when carrier gas containing the target molecule is supplied to the plurality of the molecular sensors; and
acquire a differential data between the ΔF_{SX} and the ΔF_{S} as data ΔF_{SY} of an apparent response signal.
